(19) United States
(12) Patent Application Publication
(10) Pub. No.: US 2011/0171642 A1
(43) Pub. Date: Jul. 14, 2011

(54) Analysing Methylation Specific PCR by Amplicon Melting

(75) Inventors: Alexander Dobrovic, Victoria (AU); Lasse Sommer Kristensen, Aarhus (DK)

(73) Assignee: Peter MacCallum Cancer Institute, East Melbourne Victoria (AU)

(21) Appl. No.: 12/920,080

(22) PCT Filed: Feb. 27, 2009
(86) PCT No.: PCT/AU2009/000237

§ 371 Date: Mar. 28, 2011

(30) Foreign Application Priority Data
Feb. 27, 2008 (AU) 2008900957

(51) Int. Cl. C12Q 1/68

(52) U.S. Cl. 435/6.11; 435/6.12

(57) ABSTRACT

The present invention provides a method of evaluating DNA methylation in a sample. The method comprises (i) reacting the DNA with an agent that differentially modifies methylated cytosine and non-methylated cytosine to produce modified DNA, (ii) amplifying the modified DNA by methylation specific PCR to produce amplified DNA, and (iii) subjecting the amplified DNA to melting analysis. In the method the methylation specific primers are selected such that the sequence between the primers includes a region of known sequence variation and/or at least one cytosine nucleotide.

### FIELD OF THE INVENTION

The present invention relates to methods of assaying nucleic acid methylation typically DNA methylation and to methods of diagnosis and prognosis of disorders related to aberrant methylation such as cancer or chronic disease. The method may also be used to detect foetal DNA in the maternal circulation. Using methods including methylation specific PCR and amplicon melting analysis information about the region between primers used in the PCR can be readily obtained without sequencing. The invention further provides for an assay where false positives can be readily identified to enable improved and more efficient diagnosis, monitoring, early detection or identification of predisposition to these disorders.

### BACKGROUND OF THE INVENTION

The correct establishment and maintenance of DNA methylation patterns is essential for normal development. Different cell types have characteristic methylation patterns. Aberrant DNA methylation patterns are one of the hallmarks of cancer. In most cases, promoter methylation correlates with gene silencing. This has been shown for a wide range of tumour suppressor genes including the cell-cycle inhibitor gene CDKN2A (p16INK4a), the pro-apoptotic death-associated protein kinase gene DAPK1, the cell-adhesion gene CDH1, the retinoic acid receptor gene RARB and DNA repair genes such as BRCA1, MLH1, and MGMT.

In cancer, methylation of some promoter CpG islands can be an early event, and thus methylation shows great promise as a biomarker for early detection. Conventional methods for cancer detection are in general not capable of finding pre-neoplastic and small malignant lesions, and are thus not suitable for early detection. Molecular biomarkers in body liquids such as blood, sputum or urine that allow detection and diagnosis of tumours at an early stage would be ideal. However, in these types of samples, tumour derived material is hard to detect because of the presence of material from normal cells, and thus highly sensitive and selective methods are needed. As one example, methylation of the CDKN2A promoter has been detected in the sputum of smokers up to 3 years before they are diagnosed with cancer.

Detection of low level methylation also shows great potential in the molecular monitoring of established disease after therapy. This has already been shown to be feasible in various cancers using DNA derived from plasma or serum.

Detection of low level methylation also shows potential in the identification of patients who are predisposed to cancer or chronic disease by the monitoring of normal tissues such as but not restricted to peripheral blood or buccal mucosa.

Methylation-specific PCR (MSP) is a highly sensitive method for the detection of low level methylation, and can be sensitive to at least 0.1% methylated template. However, MSP is prone to false positive results (Aggerholm, A. and Hokland, P. (2000) Blood, 95, 2997-2999; Dobrovic, A. (2005) In Coleman, W. B. and Tsongalis, G. J. (eds.), Molecular diagnostics for the clinical laboratorian. Second ed. Humana Press, Totowa, N.J., pp. 149-160; Rand, K., Qu, W., Ho, T., Clark, S. J. and Molloy, P. (2002) Methods, 27, 114-120). MSP primers are normally designed to have one or more cytosines of CpG sites at or near the 3′ end. This makes the primers highly selective for methylated template, but also facilitates amplification of incomplete converted sequences in the bisulfite treated DNA. It is thought that bisulfite treatment, in spite of recent improvements in this area, still remains the main source of variability in the analysis of DNA methylation. Recent results show that incomplete conversion may typically be in the order of 2%, even when a commercial kit is used. This variability cannot only lead to false positive results, but can also impair quantitative assays in a way that leads to overestimation of methylation levels, especially when looking at low level methylation.

Different methods for the detection of incompletely converted products co-amplified during the PCR have been proposed. These methods are relatively labour intensive and require removal of the PCR product from the tube for further analysis creating the potential for PCR contamination, or the use of additional probes as in the ConLight-MSP methodology (Rand et al, 2002, op cit.).

MSP has been made quantitative by the use of fluorescent TaqMan probes enabling real time detection of MSP products (such as in the MethyLight technique). This also eliminates any signal from non-specific amplification. However, the introduction of a probe complicates assay design, and can result in some heterogeneously methylated sequences that would otherwise be detected by MSP being missed, because of the need for the probe to hybridise correctly before a signal is observed.

Quantitative MSP using the double stranded DNA binding dye SYBR GREEN has also been described. This dye is however (1) not compatible with HRM and (2) provides less accurate quantitative data.

It is therefore desirable to provide a DNA methylation assay system to overcome many of the problems of the prior art and to provide an accurate quantification of the extent of methylation. It is also desired to identify the false positives and to understand the methylation status of the region between the MSP primers.

### SUMMARY OF THE INVENTION

The present invention provides a method for detecting and determining methylation in a nucleic acid sample and is particularly suitable for the analysis of low level methylation, said method comprising producing a methylation specific PCR (MSP) product from the sample and analysing the methylation in the sample in combination with an analysis of melting of the MSP product which method utilizes information from a region between primers used in the PCR to provide information not obtainable by electrophoresis.

In one aspect, the present invention provides a method of evaluating DNA methylation in a sample, the method comprising (i) reacting the DNA with an agent that differentially modifies methylated cytosine and non-methylated cytosine to produce modified DNA, (ii) amplifying the modified DNA by methylation specific PCR to produce amplified DNA wherein the methylation specific primers are selected such that the sequence between the primers includes a region of known sequence variation and/or at least one cytosine nucleotide, and (iii) subjecting the amplified DNA to melting analysis. In the usual case, the use of a saturating double strand binding fluorescent dye enables both the quantitation of the methylated DNA relative to a standard and the high resolution melting analysis. Typically the known sequence variation will be a SNP.

This invention provides probe-free quantitatively accurate data. Melting examination of the amplicon provides information that would not be available from standard gel electrophoresis. This method provides an analysis of the fragment by melting of any type to determine non-specific amplification, incomplete conversion, heterogeneous methylation or the presence of a variant sequence. HRM is particularly useful for the determination of the melting properties of the nucleic acid, typically DNA sample.

The type of primers and the position of the primers during PCR is a crucial part of the optimal implementation of the assay. Depending on amplicon design and primer design, especially with regards to primer placement, different types of information can be obtained from the melting analysis.

DNA double-stranded intercalating dyes may be used for analysis of DNA (or cDNA) which enables MSP analysis by real time monitoring of amplification and sensitive melting analysis of the amplicon. Dyes which do not interfere with the PCR amplification of DNA with MSP primers when used at saturating concentrations may be used.

The method of detecting and determining methylation levels in a nucleic acid, typically a DNA sample may be applied to the diagnosis and prognosis of genetic and/or chronic and/or neoplastic disorders such as cancer, cardiovascular disease, inflammatory conditions and degenerative diseases. Imprinting disorders may also be diagnosed and may be selected from the group including but not limited to Prader-Willi syndrome, Angelman syndrome, and Beckwith-Wiedemann syndrome. Other non-disease related applications can also be envisaged such as the detection of foetal DNA n the maternal circulation. The method also has potential application in non-human species.

### FIGURES

FIG. 1 shows a schematic overview of SMART-MSP. Bisulfite modified DNA is amplified in real time, in this case using a high resolution melting (HRM) compatible intercalating dye to obtain quantitative data. After real time PCR, a high resolution melting step is performed for quality control of the amplicon. The interpretation is made by considering both the real time PCR and the melting information. Two different types of SMART-MSP amplicon design are shown here, in combination with the melting profiles and amplification data that can be expected (vertical rows) in different methylation and conversion situations (horizontal rows). Incomplete conversion can be detected most readily when non-CpG cytosines are found in between the primers and no CpG cytosines are found. By including CpG cytosines in between the primers, it can be determined if the region is heterogeneously methylated or unmethylated. If the CpG cytosines in between the primers are unmethylated, the amplification might be a result of false priming. N and M are theoretical temperatures dependent on the amplicon size and sequence.

FIG. 2 shows melting profiles of a true positive result for each SMART-MSP assay. Universally methylated template was amplified and analysed by HRM analysis. Each assay has a characteristic melting profile. A: The CDH1 assay. B: The DAPK1 assay. C: The CDKN2A assay. D: The RARE assay.

FIG. 3 shows the sensitivity of the SMART-MSP assays. In all assays, the 0.1% methylated standard could be detected with high reproducibility. A: The CDH1 assay. B: The DAPK1 assay. C: The CDKN2A assay. D: The RARB assay.

FIG. 4 shows the quantitative accuracy of the SMART-MSP assays. The quantitative accuracy of the SMART MSP technology was assessed using the 2^{(-delta delta CT)} quantification approach. For each assay the calculated gene/control ratio for each standard is plotted against the dilution factor in a double logarithmic diagram. All assays proved to be quantitatively precise. A: The CDH1 assay. B: The DAPK1 assay. C: The CDKN2A assay. D: The RARB assay.

FIG. 5 shows validation of the conversion control in the DAPK1 and CDKN2A assays. A peripheral blood control sample was bisulfite treated using different times of conversion (20 min, 40 min, normal protocol), and used to test the conversion control of these assays. A: The DAPK1 assay. A gradual right-shift (increase in melting temperature) of the melting peaks was observed as the treatment time decreases. The observed right-shift of the incompletely treated samples indicates that some of the non-CpG cytosines in between the primers were not converted. Thus these samples could be identified as false positives. B: The CDKN2A assay. The 40 min treated sample and the 20 min treated sample both showed right-shifted melting peaks. Again, indicating that some of non-CpG cytosines in between the primers were not converted, and thus these samples could also be identified as false positives.

FIG. 6 shows detection of false priming from a whole genome amplified template. An assay that selected poorly against unmodified templates was used. In this assay five non-CpG cytosines and no CpG sites are found in between the primers. These non-CpG cytosines were converted to uracil in the bisulfite modified template, but not in the unmodified template. Thus, a significant right-shift of the melting profile of the unmodified amplicon is observed as none of the cytosines between the primers were converted to uracil. A: Real time PCR amplification data. B: First derivative melting peaks.

FIG. 7 shows identification of false positives in the CDH1 SMART-MSP assay. The CDH1 SMART MSP assay was performed with an additional 10 cycles to obtain amplification from the unmethylated control shown in green. A: Real time PCR amplification data. B: The melting peak of the fully unmethylated control was left-shifted by approximately 1.2° C. relative to melting peaks of the standards containing methylated template, and could thus be identified as a false positive result.

FIG. 8 shows screening of cell lines for CDH1 methylation and breast cancer samples for RARB methylation. A: CDH1 SMART-MSP amplification data for the positive cell lines. Five out the 14 cell lines screened were shown to be methylated at the CDH1 promoter. B: CDH1 MethyLight amplification data from the positive cell lines. The data from the MethyLight assay was consistent with the data from the SMART-MSP assay. C: RARB SMART-MSP amplification data for the positive tumour samples. Six out the 24 samples screened were shown to be methylated at the RARB promoter. D: RARB MethyLight amplification data from the positive tumour samples. The data from the MethyLight assay was consistent with the data from the SMART-MSP assay.

FIG. 9: Map of the MGMT promoter showing the location of primers and probes. The sequence shown starts at the transcription start site, continues through the proximal 5′ UTR into the beginning of the first intron. Vertical lines indicate CpG dinucleotides. An asterisk indicates the position of the SNP. The positions of the primers (arrows) flanking the SMART-MSP and MethyLight amplicons are indicated as well as the position of the MethyLight probe (|-|).

FIG. 10: Bisulfite sequencing of the antisense region of the MGMT promoter flanking the rs16906252 SNP. The SNP is indicated by a bold red R (G or A alleles). Conversion is essentially complete as can be seen at the blue T residues (C residues prior to bisulfate conversion). A. Completely methylated control (G allele at the SNP). B. MSP product from a heterozygous individual (A allele at the SNP). C. MSP product from a homozygous TT (AA in antisense) individual.

### DETAILED DESCRIPTION OF THE INVENTION

Methylation specific PCR (MSP) is a widely used method for the detection of DNA methylation. It uses primers specific for methylated (and optionally unmethylated), bisulfate modified DNA. MSP is based on the principle that primers with mismatched 3′ ends will not be capable of extension during the PCR. If a band that corresponds to the amplicon size given by the MSP primers is seen on a gel after PCR, it is concluded that the sample is methylated. MSP is possibly the most sensitive non-quantitative technique available and can detect 0.1% methylation or less.

A major drawback of MSP is its susceptibility to false positives. This can be due to incomplete conversion of the template, false priming or a sensitivity issue due to the capacity of MSP to amplify very low level methylation.

Accordingly in a first aspect of the present invention there is provided a method for detecting and determining methylation in a nucleic acid, typically DNA sample, said method comprising producing a methylation specific PCR (MSP) product from the sample and analysing the methylation in the MSP product in combination with an analysis of melting of the MSP product which method utilizes information from a region between primers used in the PCR to obtain information not given by electrophoresis.

This invention provides an examination of the amplicon product to provide information that would not be available from standard gel electrophoresis which merely sorts fragments on size. This method provides an analysis of the fragment by melting of any type to determine incomplete conversion or heterogeneous methylation.

This probe-free quantitative methylation specific PCR (MSP) assay incorporates evaluation of the amplicon derived from PCR in combination with melting analysis such as, but not limited to high resolution melting (HRM) analysis. This new approach to quantitative methylation detection is conveniently called Sensitive Melting Analysis after Real Time (SMART) MSP or SMART-MSP and will be referred to throughout this specification.

SMART-MSP provides information that cannot be obtained by electrophoresis, and thus functions as a quality control to avoid false positive results caused by incomplete conversion or false priming due to less stringent PCR conditions. Primer dimers or non-specific products can be detected as well.

Compared to other assays for determining DNA methylation such as MethyLight technology, SMART-MSP does not require probes.

The combination of the MSP with that of melting analysis such as HRM desirably incorporates the use of a DNA double stranded intercalating dye which then enables MSP quantitation or analysis of real time monitoring of amplification. With the use of these dyes which preferably do not inhibit PCR when they intercalate into double stranded DNA at saturating levels (Gudnason H, Dufva M, Bang D D, Wolff A. Nucleic Acids Res. 2007; 35(19):e127) highly accurate quantification, and further analysis of the amplicon by a melting analysis such as high resolution melting (HRM), has become possible by this invention. Without being limited by theory, as the temperature increases during the melting step, and the DNA ‘melts’, the dye is released and the signal of fluorescence decreases rapidly. This change in fluorescence is sequence specific and can be monitored by appropriately designed instrumentation.

Methylation Specific PCR (MSP) Product

The product arises from initial modification of nucleic acid, typically DNA by a modifying agent such as sodium bisulfite, which is converts unmethylated, but not methylated, cytosines to uracil, and subsequent amplification with primers specific for methylated versus unmethylated nucleic acid.

Nucleic Acid Samples

The nucleic acid referred to in the present invention may be selected from the group including, but limited to DNA, RNA, mRNA or any form of nucleic acid that can form a template for PCR. Typically, DNA is used. The nucleic acid, typically DNA may be derived from any biological sample which contains nucleic acid such as but not limited to blood, sputum, urine, plasma, serum, cells, fresh and archival tissues, saliva, tears, vaginal secretions, lymph fluid, cerebrospinal fluid, amniotic fluid, mucosal secretions, peritoneal fluid, ascites, fecal matter, and body exudates. The tissues may be selected from the group comprising but not limited to eyes, intestine, kidneys, brain, heart, prostate, lungs, breast and liver, histological slides and any combination thereof.

A person skilled in the art would be capable of using known methods of extracting the nucleic acid in a form that can be treated by the modifying agent to convert unmethylated, but not methylated, cytosines to uracil, and subsequent amplification with primers specific for methylated versus unmethylated nucleic acid. The nucleic acid will be in form that can be amplified.

Primers

The type of primers and the position of the primers is crucial to the optimal implementation of the assay. Depending on amplicon design and primer design especially with regards to placement, different types of information can be obtained from the melting analysis. This information cannot be obtained by electrophoretic analysis. In particular, identification of false positives due to incomplete bisulfite conversion or false priming is possible. Heterogeneous methylation can also be distinguished from homogeneous methylation. The presence or absence of given alleles of a sequence variation can also be determined

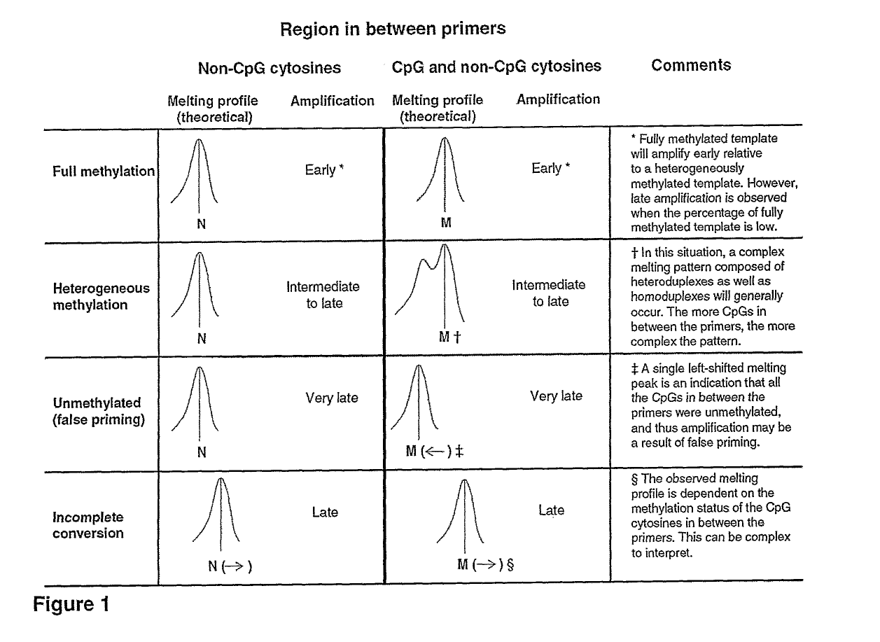

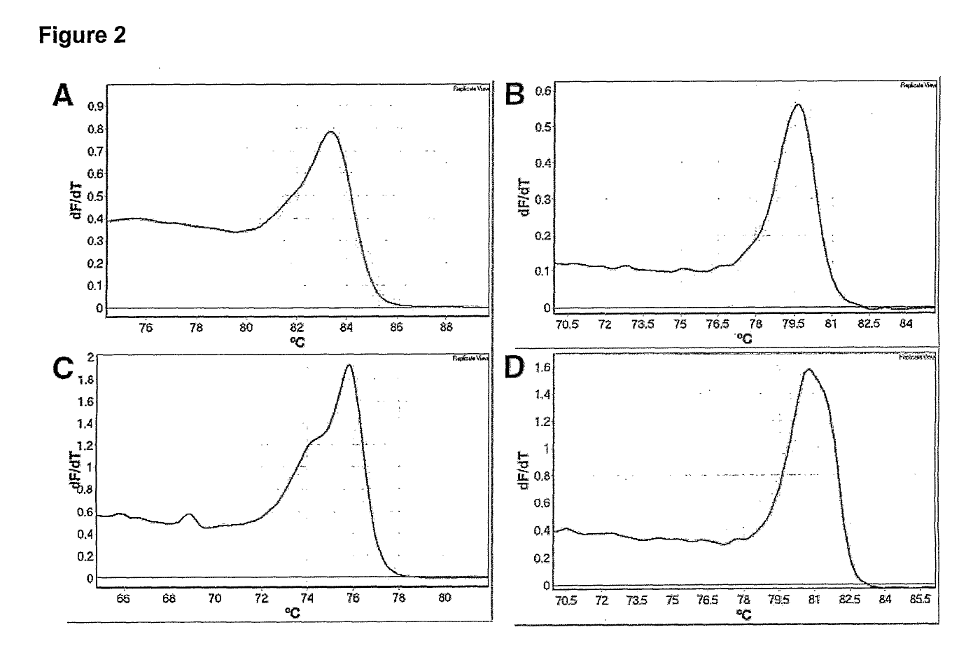

SMART-MSP can give accurate quantitative data typically for DNA methylation detection. The combination of MSP with melting analysis enables the sensitive screening of the region in between the MSP primers. Several types of primer positioning may be used including: (a) only non-CpG cytosines between the primers allowing assessment if (low) levels of amplification are due to incomplete conversion or (b) CpGs (with as few non-CpG cytosines as possible) between the primers allowing assessment if (low) levels of amplification are due to partial or heterogeneous methylation; or (c) only CpG cytosines between the primers or (d) a sequence variant in between the primers. Interpretation is made by considering both the quantification and the melting information (FIG. 1). When the melting profile of a true positive is established (FIG. 2) there is usually no need for gel electrophoresis analysis or any further processing, and thus SMART-MSP is a closed-tube method.

HRM analysis can easily detect single base pair changes in the amplified DNA sequence. Thus, by including non-CpG cytosines between the primers, the conversion status of these can be assessed. If cytosines in between the primers are not converted, the amplicon will melt late relative to amplicons derived from fully converted template (FIG. 1).

The use of a melting analysis such as HRM can give information about the methylation status of CpGs between the primers, but the melting analysis step can also be used as a control to indicate amplification of incompletely modified sequences, false priming or non specific products. Thus SMART-MSP is less prone to false positive results and overestimation of methylation levels.

Dyes

When a melting analysis such as HRM is used, HRM compatible DNA double stranded intercalating dyes which enable MSP quantitation by real time monitoring of amplification are desirably used. The dyes are added to the MSP. In one embodiment, the dyes are used at saturating concentrations which do not interfere with the PCR reaction for the amplification of DNA with MSP primers. This enables the melting analysis methodology to be used with the MSP technology. Dyes having these characteristics and which are useful for this assay include but are not limited to SYTO®9, EvaGreen™, and LC Green®.

Quantification and Analysis

Real time amplification of bisulfate converted nucleic acid, typically DNA with MSP primers is performed with a fluorescent dye, which does not inhibit the PCR when used at saturating conditions. This allows for highly accurate quantitative results to be obtained without the use of probes and for melting analysis to be performed. Quantification is based on cycle threshold (Ct) values, and thus it is desirable to run a control assay in parallel to normalise for the amount of input nucleic acid, typically DNA in the PCR.

In the SMART-MSP methodology, representing one embodiment of the invention, sensitive melting analysis is performed immediately after the real time PCR generally in a closed-tube system. The kind of information that can be obtained from HRM is dependent on amplicon design and placement, and is typically interpreted by considering the amplification data as well (FIG. 1). Firstly, when only non-CpG cytosines are included in between the primers, the melting step can function as a control for amplification of incompletely converted DNA. If incompletely converted sequences are amplified, the amplicon will have a higher GC-content relative to the fully converted amplicons, and will therefore melt later.

When CpG sites are included between the primers, the melting step can be used to assess if these CpG sites are methylated. Generally, if left-shifted melting (indicating a significantly decreased melting temperature) is observed relative to the fully methylated control, this is an indicator that some or all of the CpG sites are not methylated. A complex melting pattern consisting of heteroduplexes as well as homoduplexes can occur if more than one molecule is amplified during the PCR and the studied region is heterogeneously methylated. Thus, a heterogeneously methylated region can give a melting profile extending to the left, due to the melting of molecules with different CpG positions being methylated and heteroduplex formation between them (FIG. 1). Left shifting can also indicate false positives due to false priming. Generally, false priming is associated with very late amplification (FIG. 1). False priming can be minimised by stringent PCR conditions. Assays are typically designed to include as few non-CpG cytosines as possible, preferably none. A conversion control can be performed in parallel when non-CpG cytosines cannot be avoided to be confident that these are converted.

In other assays, quantification is based on comparisons with melting profiles of a standard dilution series that need to be included in every run. This is not necessary when performing SMART-MSP assays which quantify relative to a 100% methylated control and the amplification of a CpG-free control sequence.

The information obtained from the melting analysis may be analysed by methods known to the skilled addressee. Standard analytical tools used for melting analysis can be applied to the MSP product.

Advanced Data Processing

The method of the present invention may be used for the high throughput methylation analysis of nucleic acid, typically DNA samples. Therefore, the invention also involves analysis of data using a computing device. In a preferred embodiment the device may comprise one or more databases. In a further preferred embodiment the device may comprise one or more learning algorithms.

Diagnosis and Prognosis of Conditions Related to DNA Methylation

Cancer treatments, in general, have a higher rate of success if the cancer is diagnosed early and treatment is started early in the disease process. The relationship between improved prognosis and stage of disease at diagnosis hold across all forms of cancer for the most part. Therefore, there is an important need to develop early assays of general tumorigenesis that measure general tumorigenesis without regard to the tissue source or cell type that is the source of a primary tumor. Moreover, there is a need to address distinct genetic alteration patterns that can serve as a platform associated with general tumorigenesis for early detection and prognostic monitoring of many forms of cancer.

Accordingly in another aspect of the present invention there is provided the use of the method described for the diagnosis and prognosis of genetic and/or chronic and/or neoplastic disorders such as cancer, cardiovascular disease, inflammatory conditions and degenerative diseases. Imprinting disorders may also be diagnosed and may be selected from the group including but not limited to Prader-Willi syndome, Angelman syndrome, and Beckwith-Wiedemann syndrome. The invention provides a method of diagnosis or prognosis of a genetic disorder said method comprising determining a level of methylation of a nucleic acid, typically DNA sample from a patient and concluding the diagnosis or prognosis of the genetic disorder from the level of methylation. The method may also be used to detect foetal DNA in the maternal circulation.

The application of the method of detecting and determining nucleic acid, typically DNA methylation can be applied to any situation in which a diagnosis or prognosis relies on an accurate determination of nucleic acid, typically DNA methylation. The present invention is not limited to the described uses for diagnosis or prognosis.

In the present invention, diagnosis and prognosis is intended to mean and include but is not limited to prediction of a predisposition to, or a diagnosis of, or prognosis of, or monitoring of, or determining the likely response to clinical intervention of a genetic and/or chronic, and/or neoplastic, disorder.

Detection of low level methylation also shows potential in the identification of patients who are predisposed to cancer or chronic disease by the monitoring of normal tissues such as but not restricted to peripheral blood or buccal mucosa.

DNA methylation patterns may be determined in a wide range of tumour suppressor genes including but not limited to the cell-cycle inhibitor gene CDKN2A (p16INK4a), the pro-apoptotic death-associated protein kinase gene DAPK1, the cell-adhesion gene CDH1, the retinoic acid receptor gene RARB and DNA repair genes such as //BRCAI, MLH1, WRN and MGMT. However, a person skilled in the art will be able to determine the appropriate genes which correlate to the genetic and/or chronic and/or neoplastic disorders to analyse for their diagnosis or prognosis.

In cancer, methylation of some promoter CpG islands can be an early event, and thus the detection of methylation shows great promise as a biomarker for early detection. The assay provided in this invention allows for the early detection of aberrant DNA methylation.

Detection of low level methylation also shows great potential in the molecular monitoring of established disease after therapy. Hence the use of the assay can assist in the prognosis and monitoring of the progression of the disease.

Thus in one embodiment the method would involve a first step of obtaining a tissue or blood sample from a patient from which a nucleic acid such as DNA can be extracted. The sample would undergo chemical treatment with a compound such as bisulfite to convert unmethylated, but not methylated cytosines to uracil. An amplification step utilising primers (designed and placed appropriately) would be conducted in the presence of a dye that is preferably HRM compatible to provide an MSP product. This product would then be available to be analysed preferably by HRM to determine a level of methylation. This level of methylation can be compared against a control or known levels of methylation that are indicative of a particular genetic disorder.

Similarly, the level of methylation can be indicative of the progression of a genetic disorder. Higher levels of methylation may be an indication that the disorder is progressing.

Other applications may be adopted using the methods of determining nucleic acid typically DNA methylation levels. For instance the determination of the propensity to chronic disease or cancer may be applied when compared against suitable controls. Individuals with above normal levels of methylation either at one specific locus or at a panel of loci may be considered at higher risk of developing subsequent disease.

Kits

Another aspect of the present invention is a kit for conducting an assay according to the methods described herein, comprised of a reagent containing bisulfite, primers and additional oligonucleotides for the production of amplified products, and a nucleic acid intercalating dye and optionally, instructions and additional components for conducting at least one of the described variants of the method.

Throughout the description and claims of this specification, the word “comprise” and variations of the word, such as “comprising” and “comprises”, is not intended to exclude other additives, components, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

The present invention will now be more fully described with reference to the accompanying examples and figures. It should be understood, however that the description following is illustrative only and should not be taken in any way as a restriction on the generality of the invention described above.

### EXAMPLES

### Example 1

### The Sensitivity and Quantitative Accuracy of the SMART-MSP Assays

Assays have been developed for the promoter regions of the CDH1, DAPK1, CDKN2A (p16INK4a), and RARB genes as proof of principle. This example shows that highly accurate quantification is possible in the range from 100% to 0.1% methylated template when 25 ng of bisulfite modified DNA is used as a template for PCR.

(a) Samples and DNA Extraction.

Purified genomic DNA from cell lines (2008, MCF7, Hs578T, MCF10A, MDA-MB-468, MDA-MB-231, MDA-MB-435, PC3, SKBr-3, Colo205, RPMI8226, SW948, HL-60, and T47D) was used. Universal Methylated DNA (Chemicon, Millipore, Billerica, Mass.) was used as a fully methylated positive control. DNA from peripheral blood mononuclear cells from normal individuals was used as unmethylated DNA for dilutions. Standard dilution series of 100%, 10%, 1%, 0.1% and 0.01% methylation levels were prepared by diluting the fully methylated DNA into the unmethylated DNA.

(b) Bisulfite Modification

Five hundred ng of genomic DNA product was subjected to bisulfite conversion according to the manufacturer's instructions. Incomplete bisulfite modified DNA was prepared by treating the appropriate samples for 20 min or 40 min with the bisulfite mix solution and purified as described above.

(c) SMART-MSP Primer Design

The primers were usually designed to include at least 2 CpG sites within each of the primer sequences, and one of the cytosines of a CpG site was usually placed at or as close as possible to the 3′ end. This will make the primers as selective for methylated templates as possible, and ensure that only methylated templates are amplified during PCR when sufficiently stringent conditions are chosen. Non-CpG cytosines were included in the primer sequences as well to select against incomplete converted sequences, and at least one of these was placed as close to the 3′ end as possible. The primer sequences and genomic regions spanned, as well as amplicon size and the annealing temperatures (TA) can be found in Table 1.

TABLE 1
Primer sequences, annealing temperatures, and amplicon information for typical SMART-MSP assays

| | | | Amplicon | CpGs/non- | | |
|---|---|---|---|---|---|---|
| | Primer sequences (CpG sites | Annealing | size | CpG Cs | | |
| | in bold and converted Cs | tempera- | (base | between | | Publica- |
| Gene | as capital Ts or As | ture | pairs) | primers | Spanned region | tions |
| | Non-CpG C's only between the | | | | | |
| | primers | | | | | |
| CDKN2A | F-gTaTTtTTtTcgagTaTtcgTtTacggc | 63° C. | 72 | 0/6 | 21964971-21965042 | K08 |
| | R-caaatcctctAAaAAAaccgcgA | | | | of Chr. 9 | |
| DAPK1 | F-aggaTagTcggaTcgagTTaacgTc | 67° C. | 61 | 0/4 | 89302618-89302678 | K08 |
| | R-ttAccgaAtcccctccgcgA | | | | of Chr. 9 | |
| APC | F-tTcgTtggatgcggaTTagggc | 68 | 55 | 0/7 | 112101367-112101421 | K09 |
| | R-ccaatcgAcgAActcccgacg | | | | of Chr. 5 | |
| CDH1 | F-gtgggcgggTcgtTagTtTc | 65/68 | 58 | 0/3 | 67328555-67328612 | K09 |
| | R-AccacaAccaatcaAcaAcgcgA | | | | of Chr. 16 | |
| GSTP1 | F-gcgaTtTcggggaTtTTagggc | 67 | 51 | 0/5 | 67107679-67107729 | K09 |
| | R-tAcaccccgAAcgtcgAccg | | | | of Chr. 11 | |
| HIC1 | F-TTaggcggTTagggcgTcgTac | 66 | 54 | 0/4 | 1906662-1906714 | K09 |
| | R-ctAcgAAAacacacaccgAccgA | | | | of Chr. 17 | |
| RARB | F-atgTcgagaacgcgagcgatTc | 71 | 64 | 0/3 | 25444860-25444923 | K09 |
| | R-gttccgAatcctaccccgacgA | | | | of Chr. 3 | |
| RASSF1A | F-cgTTcggTTcgcgTttgTtagc | 68 | 58 | 0/5 | 50353240-50353297 | K09 |
| | R-tAAcccgAttAAAcccgtActtcg | | | | of Chr. 3 | |
| TWIST1 | F-cgcggTTaggaTagtTtTTtTcgaTc | 68 | 59 | 0/4 | 19124102-19124160 | K09 |
| | R-aAcgcccccgaaccctaAcg | | | | of Chr. 7 | |
| MGMT | F-TTcggatatgTtgggaTagTTcgc | 67 | 54 | 0/4 | 131155463-131155516 | C09 |
| | R-gAAcgtcgAAacgcaaaAcg-3′ | | | | of Chr 10 | |
| BRCA1 | tgTttagCGgtagTTTTttggtttTC | 65 | 49 | 0/1 | 38530948-38530996 | unpublished |
| | ttccCGCGcttttcCG | | | | of Chr. 17 | |
| MLH1 | CGTtgaagggtggggTtggatggC | 63 | 70 | 0/3 | 37009913-37009982 | unpublished |
| | cacctcaAtAcctCGtActcaCGttct | | | | of Chr 3 | |
| miR-663 | cGgggggtTTtTtgaCGCGgTa | 65 | 57 | 0/5 | 26138243-26138299 | unpublished |
| | AtCGacaaccacaAAaAACGaAaACG | | | | of Chr 20 | |
| | tgCGCGTagCGttTCGtTCGgC | 65 | 64 | 0/6 | 26137155-26137218 | |
| | AAaACGAACGAaAaActAactCGCGACGA | | | | of Chr 20 | |
| | Both CpG C's and non-CpG C's | | | | | |
| | between the primers | | | | | |
| CDH1 | F-gtgggcgggTcgtTagTtTc | 64° C. | | 2/8 | 67328555-67328640 | K08 |
| | R--cgctAattAActAaAAAttcacctAccg | | | | of Chr. 16 | |
| RARB | F-TcgagaacgcgagcgatTc | 63° C. | 146 | 5/18 | 25444864-25445008 | K08 |
| | R-gAccaatccaAccgAAAcg | | | | of Chr. 3 | |
| MLH1 | ATcgcgGcgGGGGAAGTTATttAGc | 70 | 95 | 1/6 | 37010097-37010191 | unpublished |
| | ccttaaAtAaAccCGActCGactccctcC | | | | of Chr 3 | |
| | Amplicons flanking a poly- | | | | | |
| | morphism sequence variant | | | | | |
| BRCA1 | TGGCGTGGGAGAGTGGATTTtC | | 98 | 2/5 | 38530664-38530761 | unpublished |
| MSP SNP | R-CCCAaaaTTCACAACGCCTTACG | | | | of Chr 17 | |
| MGMT | ggTtgTTaTCGtTTCGagggagagTt | 67 | 53 | 0/0 | 131155509-131155561 | C09 |
| SNP | cgcgCCCcgaATATaCTaaaAC | | | | of Chr 10 | |
| | Control genes | | | | | |
| HMBS | F-GGTTTGATTTTTTGTTTTAGGGTTATT | 60 | 59 | 0/4 | 118465750-118465808 | unpublished |
| | R-TACCACCAATCAACACTCCTCAAA | | | | of Chr 11 | |
| COL2A1 | F-gTaatgTTaggagTaTTTtgtgggTa | 65° C. | 86 | N/A | 46667210-46667295 | K08 |
| | R-ctaccccaAAaAaAcccaAtcctA | | | | of Chr. 12 | |

Publications:

K08: Kristensen, L S, Mikeska T, Krypuy M, Dobrovic A. Sensitive Melting Analysis after Real Time-Methylation Sensitive PCR (SMART-MSP): high-throughput and probe-free quantitative DNA methylation detection. Nucleic Acids Research 2008 April; 36(7):e42. Epub 2008 Mar. 15.

K09: Kristensen, L S, Raynor M, Dobrovic A. Methylation profiling of peripheral blood mononuclear DNA from normal individuals reveals mosaic promoter methylation of cancer associated genes (in preparation).

C09: Candiloro L M, Dobrovic A. Detection of MGMT promoter methylation in the peripheral blood of normal individuals is strongly associated with the T allele of the rs16906252 MGMT promoter SNP. Cancer Research (submitted).

(d) PCR and HRM Conditions for the SMART-MSP Assays

PCR cycling and HRM analysis were performed on the Rotor-Gene Q (Qiagen, Hilden, Germany). SYTO® 9 was used as the intercalating dye (Invitrogen, Carlsbad, Calif.). The reaction mixtures consisted of 25 ng of bisulfite modified template (theoretical amount), 1× PCR buffer, 2.5 mmol/L MgCl₂ final (3 mmol/L in the CDH1 assay), 200 nmol/L of each primer, 200 μmol/L of each dNTP, 5 μmol/L of SYTO 9, 0.5U of HotStarTaq (Qiagen) (5U/μL) in a final volume of 20 μL. The cycling protocol started with one cycle of 95° C. for 15 min for enzyme activation, followed by 45 cycles of 95° C. for 20 s, annealing at the appropriate temperature (Table 1) for 30 s, 72° C. for 30 s, and one cycle of 95° C. for 1 min. HRM was performed from 60° C. to 90° C., with a temperature increase at the rate of 0.2° C. per second for all assays. The annealing temperature was experimentally determined for each assay to ensure only methylated templates were amplified. For each assay, a standard dilution series were run to assess the quantitative properties and sensitivity of these. Fully methylated and fully unmethylated control unmodified control and no template control were also included in every run. All samples were analysed in triplicate and each breast tumour sample in duplicate.

(e) Real Time PCR Quantification

The relative 2^{(-delta delta CT)} quantification approach (Livak, K. J. and Schmittgen, T. D. (2001) Methods, 25, 402-408) was used. The CT value of the control COL2A1 sequence (Table 1) is subtracted from the CT value of the target gene for the calibrator sample, the 100% methylated standard. For each test sample, this value is then subtracted from the value resulting from the CT value of the target gene minus the CT value for the COL2A1 control sequence. This gives the “delta delta CT” value, which is then put in to the equation, 2(-delta delta CT), and multiplied by 100 to give the percentage of methylation relative to the 100% methylated control. For this approach to be valid, the amplification efficiencies of the gene and the control must be approximately equal. The take-off values given by comparative quantification (a feature of the Rotor-Gene 6000 Series Software, version 1.7.61) were used as CT values in the calculations. The take-off point is defined as the cycle at which the second derivative is at 20% of the maximum level, which indicates the end of the background noise and the transition into the exponential phase.

Melting Profiles of the SMART-MSP Assays

The melting profiles of a true positive result, obtained by amplifying standards containing methylated template for each assay, were used as references for unknown samples (FIG. 2). Since all the amplified dilution standards for a given amplicon have the same melting profile, any one may be chosen as a reference. Confirmatory gel electrophoresis was performed in the development phase for all assays, and only one band of the expected size was observed.

Sensitivity and Quantitative Accuracy

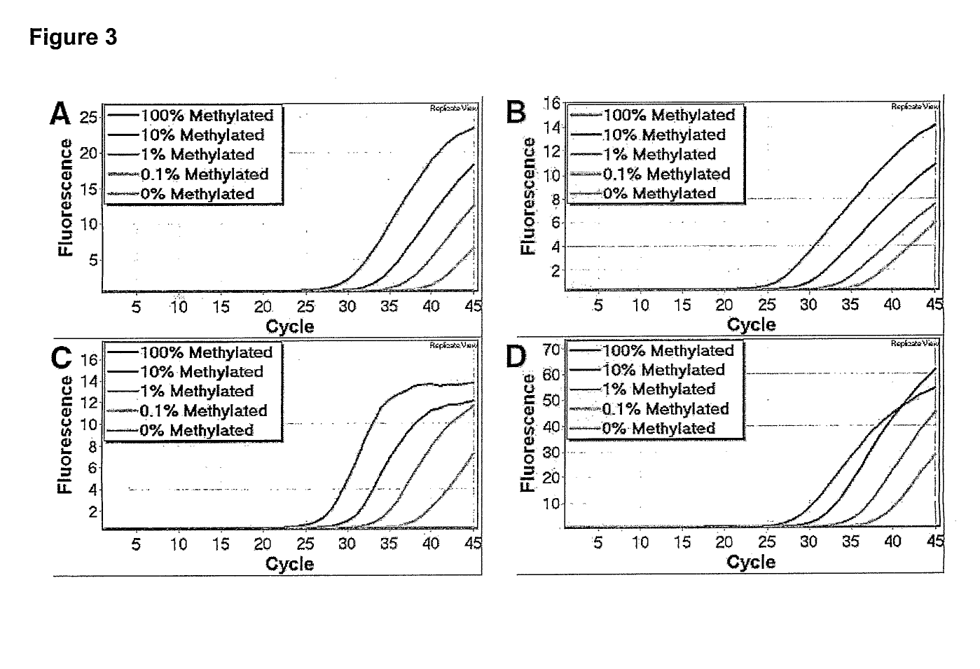

Each SMART-MSP assay was optimised so that amplification only occurred from standards containing methylated template, and no amplification was seen in the fully unmethylated control in the unmodified control or in the no template control. The sensitivity and quantitative accuracy of the assays were determined using the standard dilution series. All assays were able to reliably detect the 0.1% methylated standards (FIG. 3).

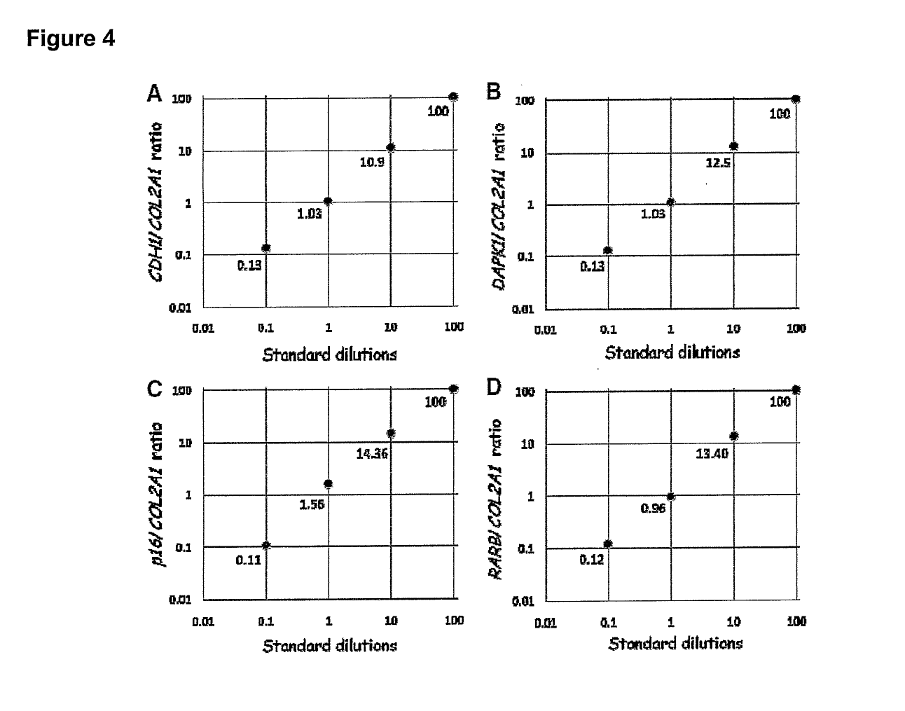

The quantitative accuracy of each assay was determined using the relative 2^{(-delta delta CT)} quantification approach. Amplification efficiencies of the gene and the control were approximately equal for all assays. The calculated values for each standard were plotted versus the dilution factor for each gene (FIG. 4).

All reactions contained approximately equal amounts of template suitable for PCR. This was evident from the similar CT values obtained in the control assays. The software to obtain a standard curve for the dilution series for calculation of the correlation coefficient (r2) for each assay was used. The correlation coefficient for each assay (DAPK1: r2=0.995, CDKN2A: r2=0.998, CDH1: r2=0.999, RARB: r2=0.995) indicated a strong linear relationship between CT values and given concentrations for all assays.

The CDH1 and RARB MethyLight assays were also tested. These MethyLight assays were quantitatively accurate in the range from 100% down to 0.1% methylated template. The correlation coefficients of the MethyLight assays were: CDH1: r2=0.984 and RARB: r2=0.983, again indicating a strong linear relationship between CT values and given concentrations for both.

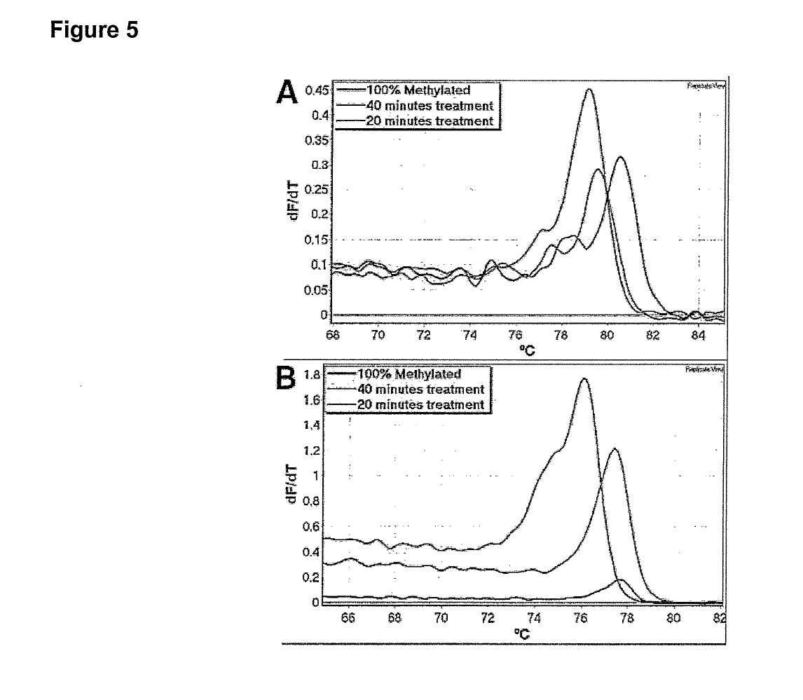

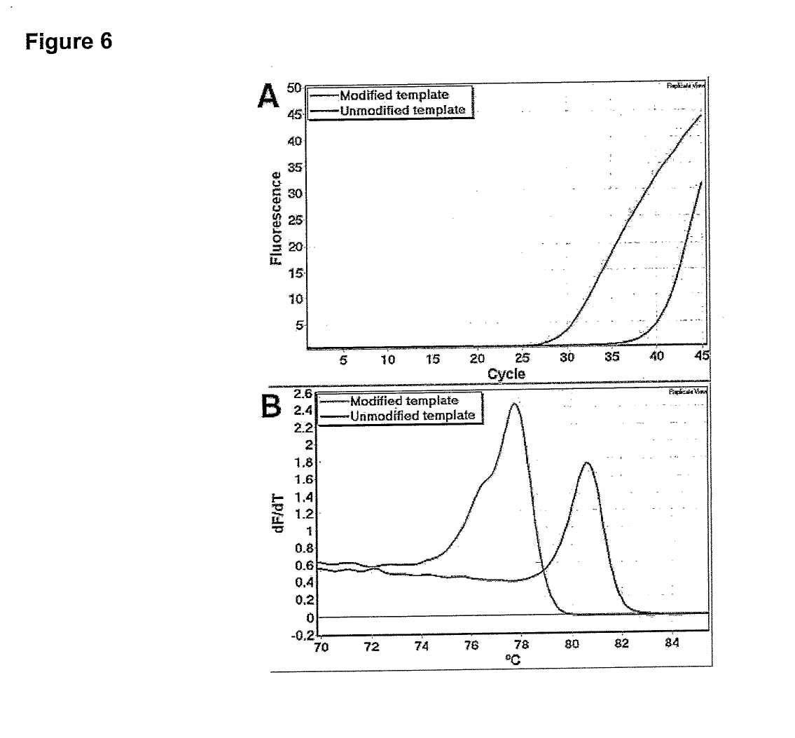

Incomplete bisulfate conversion is probably the most problematic cause of false positive results. When performing MSP experiments, it is thus important to be confident that positive results are not derived from incomplete conversion. MSP primers are normally designed to have two or preferably more cytosines deriving from CpG sites at or near the 3′ end. This makes the primers highly selective for methylated template. However, this also facilitates amplification of incomplete converted sequences in the bisulfite treated DNA as unconverted sequence resembles methylated sequence. It is possible that only a small subset of the DNA copies suffer a substantially lower conversion rate, which in combination with the high sensitivity of MSP can lead to false positives or overestimation of results. Also, it is possible that the distribution of unconverted sites is non-random, thus making some promoter regions more prone to incomplete conversion than others, and that conversion rates are dependent on DNA quality. Thus, in spite of stringent PCR conditions, a false positive result can arise if bisulfite conversion is incomplete. For these reasons, it is recommended that a control for incomplete conversion is performed. This control should preferably assess the conversion status of non-CpG cytosines in the vicinity of the primer positions. The results show that having a non-CpG cytosine at the 3′-end is not sufficient to rule out the possible amplification of incomplete converted DNA (FIGS. 5 and 6).

If the annealing temperature is too low or too many cycles are used, amplification can occur across the 3′ mismatch. This type of false positive can be detected by the use of an appropriate negative control (e.g. whole genome amplification product or cell line DNA).

Finally, the sensitivity of MSP might lead to false positives because of the amplification of a rare subpopulation of methylated sequences. The tumour sample might be extremely heterogeneous, with only a small proportion of methylated cells. Alternatively, the methylation might derive from normal cells in the tumour biopsy. In either case, it would be incorrect to call the tumour methylated for that particular gene. This problem arises because MSP is a non-quantitative methodology.

The present invention shows that SMART-MSP can give accurate quantitative data for DNA methylation detection. The combination of MSP with HRM, which is enabled by the use of a HRM compatible DNA double stranded intercalating dye, enables the sensitive screening of the region in between the MSP primers. Thus information is provided that cannot be obtained by electrophoresis. The following section has already been stated earlier. For full utility of the methodology, one or both of two types of primer positioning may be used: (a) only non-CpG cytosines between the primers allowing assessment if (low) levels of amplification are due to incomplete conversion and (b) CpGs (with as few non-CpG cytosines as possible) between the primers allowing assessment if (low) levels of amplification are due to partial or heterogeneous methylation. Interpretation is made by considering both the quantification and the HRM information (FIG. 1). When the melt profile of a true positive is established (FIG. 2) there is no need for gel electrophoresis analysis or any further processing, and thus SMART-MSP is a closed-tube method.

### Example 2

### Validation of the DAPK1 and CDKN2A SMART-MSP Conversion Control Assays

Bisulfite conversion can be assessed by melting analysis using assays with non-CpG cytosines between the primers. If a right-shift of the melting profile is observed, this can only be due to incomplete conversion of some or all of the non-CpG cytosines in between the primers or amplification of non-specific products (FIG. 1). Since incompletely converted products are of the same size as true positives, these can not be distinguished using gel electrophoresis. Incompletely converted DNA was generated to assess whether its amplification showed right-shifted melting profiles in these assays. Amplification was usually seen from these samples and always showed right-shifted melting profiles. The 100% methylated standard amplified earlier than incompletely converted DNA in both assays, and thus gave higher melting peaks (FIG. 5).

Bisulfite modified template melts early relative to unmodified template (FIG. 6). A primer pair (5′-gggaagatgggatagaagggaataT and 5′-tctAacaAttAtAAActccaaccaccaa) with a limited number of non-CpG cytosines in their sequences (shown in upper case), and thus not particularly discriminatory for methylated sequences, was used to amplify bisulfite modified as well as unmodified DNA from the same sample. In this assay, 5 non-CpG cytosines and no CpG sites are found in between the primers. Since the unmodified amplicon has a higher GC-content it is more stable and melted later than the bisulfite modified amplicon.

By generating incompletely converted DNA sequences, and using these as templates for the CDKN2A and DAPK1 assays, it was shown that amplification from these usually occurred, and were easily identified as they always corresponded to right-shifted melting peaks (FIG. 5). In addition, unmodified DNA was amplified as well as modified DNA derived from the same individual using primers that select poorly between modified and unmodified DNA, and observed highly reproducible right-shifted melting peaks from the unmodified template relative to the modified one (FIG. 6).

### Example 3

### Identification of False Positives in the CDH1 SMART-MSP Assay

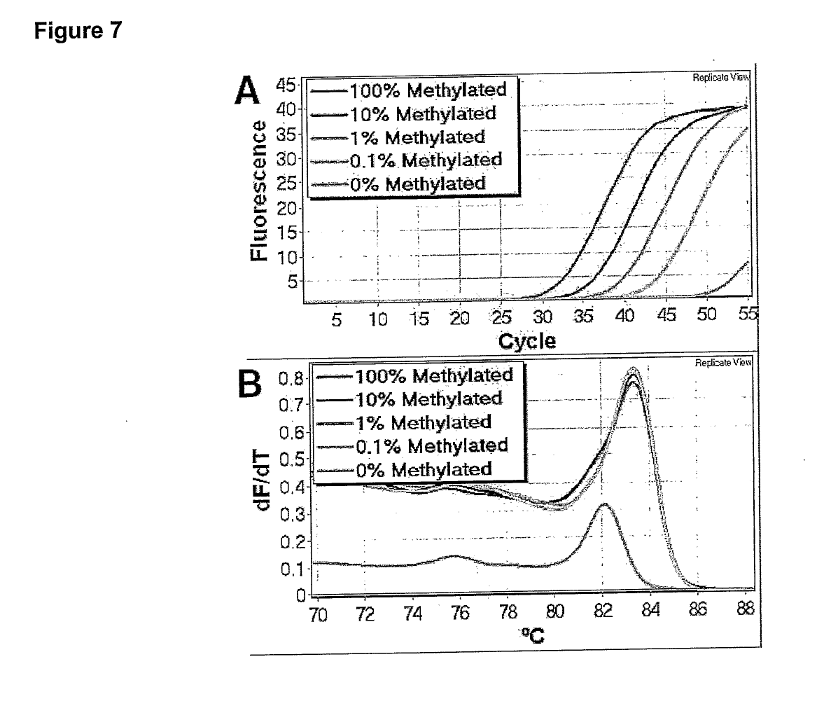

By running the CDH1 SMART-MSP assay for an additional 10 cycles, late amplification from the fully unmethylated control (WGA product) occurred. Since this WGA control is not methylated at the two CpG sites in between the primers, it was expected to see a readily distinguishable left-shifted melting peak, and thus to be able to identify it as a false positive result (FIG. 1). The melting peak of the unmethylated control (WGA product) was shifted approximately 1.2° C. to the left compared to the standards containing methylated template (FIG. 7).

False positive results due to false priming can be detected by HRM analysis as well, if CpGs are included in between the primers (FIG. 1). By running the CDH1 SMART-MSP assay for an additional 10 cycles, late amplification from the fully unmethylated control was seen. In this case, a left-shifted melting peak was observed due to the two CpGs found in between the primers being unmethylated. This allowed identification as a false positive result (FIG. 7). However, sufficiently stringent PCR conditions and adequate primer design are the best insurance against false priming.

A left-shifted peak can also be due to the target sequence being heterogeneously methylated, which can result in heteroduplex formation, and thus the melting profile will often be visually different (FIG. 1). Methodologies that utilise MSP primers are only semi-quantitative when heterogeneously methylated DNA is amplified. For this reason, MSP might be less suited in those cases when CpG islands show highly variable methylation. However, by including CpG sites in between the primers in the SMART-MSP methodology, it can be assessed whether the studied region is heterogeneously methylated or not, by the HRM analysis.

### Example 4

### Screening of Cell Lines

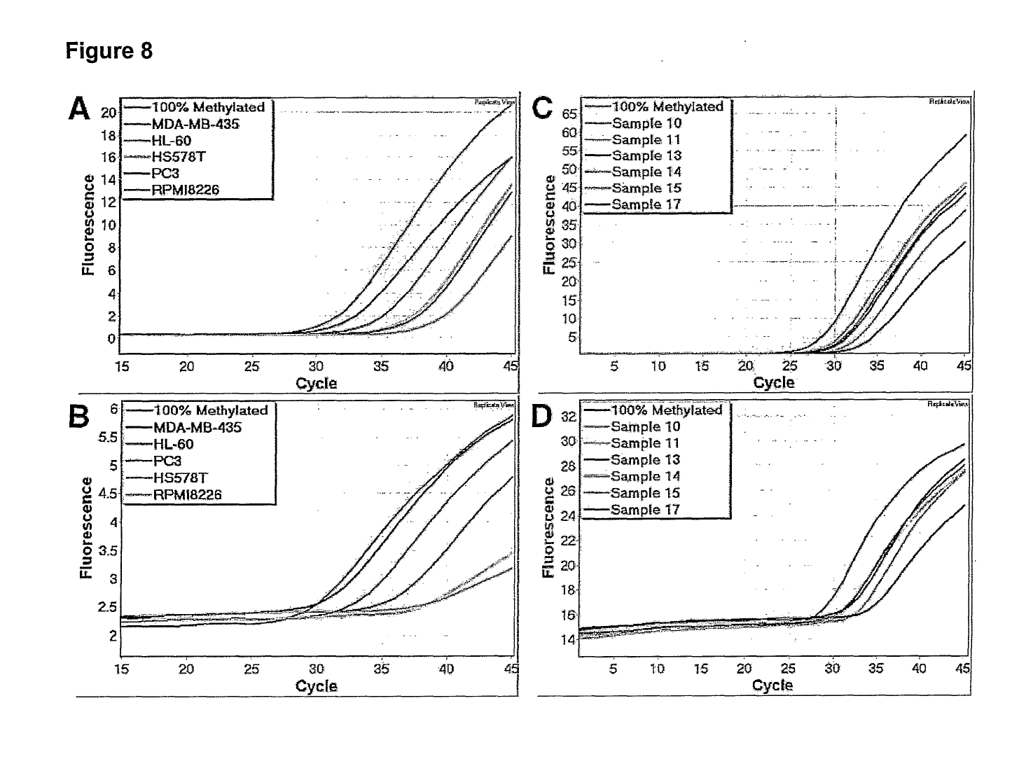

The reliability of the method was tested using a panel of 14 cell lines (2008, MCF7, HS578T, MCF10A, MDA-MB-468, MDA-MB-231, MDA-MB-435, PC3, SKBr-3, Colo205, RPMI8226, SW948, HL-60, and T47D) with the CDH1 and CDKN2A SMART-MSP assays. Five of these cell lines were shown to be methylated at the CDH1 promoter at various levels (HL-60: 100%, MDA-MB-435: 75%, PC3: 10%, HS578T: 6% and RPMI8226: 2%). The amplification data (FIG. 8A) were used to calculate the methylation levels as described. These results were validated using the CDH1 MethyLight assay, in which the same 5 cell lines were shown to be methylated at similar levels (FIG. 8B). These results were consistent with previously published data on these cell lines (Paz, M. F., Fraga, M. F., Avila, S., Guo, M., Pollan, M., Herman, J. G. and Esteller, M. (2003) Cancer Res, 63, 1114-1121; Reinhold, W. C., Reimers, M. A., Maunakea, A. K., Kim, S., Lababidi, S., Scherf, U., Shankavaram, U. T., Ziegler, M. S., Stewart, C., Kouros-Mehr, H. et al. (2007) Mol Cancer Ther, 6, 391-403).

However, the HS578T cell line was estimated to be methylated at lower levels when using the MethyLight assay relative to the SMART-MSP assay. This may be due to the fact that the probe in the MethyLight assay overlays two CpGs and that these are not consistently methylated in this cell line. The melting profiles obtained by the SMART-MSP assay was left-shifted, indicating that at least one of these two CpGs sites were unmethylated (data not shown).

Five of the 14 cell lines were shown to be methylated by SMART-MSP at various levels at the CDKN2A promoter (T47D: 65%, PC3: 35%, RPMI8226: 30%, Colo205: 1% andSW948: 0.1%). Again, these results were consistent with previously published data on these cell lines (Paz, M F et al (2003); Wong, I. H., Ng, M. H., Lee, J. C., Lo, K. W., Chung, Y. F. and Huang, D. P. (1998) Br J Haematol, 103, 168-175)

### Example 5

### Screening of Breast Cancer Samples

The diagnostic applicability of the SMART-MSP methodology was tested using the RARB and CDH1 assays on a panel of 24 breast cancer samples. 6 out of the 24 samples were found to be methylated at the RARB promoter region at levels higher than 5% (76%, 57%, 44%, 33%, 29% and 7%). The amplification data (FIG. 8C) were used to calculate the methylation levels. Data from the control assay is not shown. These results were validated using the RARB MethyLight assay, in which the same six samples were shown to be methylated at similar levels (FIG. 8D). These findings are in agreement with what has recently been reported for this locus in breast cancer (Dahl, C. and Guldberg, P. (2007). Nucleic Acids Res, Epub ahead of print; Feng W, S. L., Wen S, Rosen D G, Jelinek J, Hu X, Huan S, Huang M, Liu J, Sahin A A, Hunt K K, Bast R C Jr, Shen Y, Issa J P, Yu Y. (2007) \ Breast Cancer Res, 9, Epub ahead of print.

None of the samples showed high level methylation at the CDH1 promoter. Eleven samples were found to be methylated between 0.1% and 0.7% which could be detected with high reproducibility. Furthermore, ten samples were found to be methylated in the interval from 0.01% to 0.1%, for which the reproducibility between samples was less good. These observed values might only account for a biologically insignificant background methylation level that can be found in normal cells. A recent study has found that a low background of CDH1 methylation is present in normal breast tissue, and that this level did not differ significantly from what was found in adjacent malignant breast tissue (Feng W, S. L., et al (2007)). Another recent study reports that CDH1 methylation was not found in a cohort of 28 tissue biopsies from 17 breast cancer patients (Dahl, C. and Guldberg, P. (2007)). However, the method used in that study was not sensitive enough to detect methylation levels below 1%. Thus, if CDH1 methylation is detected using a purely qualitative and highly sensitive methodology, positive results might arise due to low background methylation.

The same samples were tested using the CDH1 MethyLight assay with comparable results. However, the CDH1 MethyLight assay was not able to detect methylation in four of the samples showing the lowest levels of methylation in the CDH1 SMART-MSP assay.

### Example 6

### Detection of MGMT Promoter Methylation

Methods

Samples

The investigations were performed after approval by the Peter MacCallum Cancer Centre Ethics Committee (Project 02/70). Eighty nine peripheral blood samples from normal blood donors were obtained after informed consent from the Australian Red Cross Blood Service.

DNA Extraction and Treatment

Mononuclear cells were extracted from peripheral blood using Histopaque-1077 as per manufacturer's instructions (Sigma Aldrich, St Louis, Mo.) and DNA was extracted using the Puregene kit (Qiagen Sciences, Germantown, Md.) omitting red cell lysis, or using the salting out method (12). DNA quantification was performed on a NanoDrop ND-1000 spectrophotometer (NanoDrop Technologies, Wilmington, Del.). DNA was diluted to 5 ng/μL in DNA hydration solution (Qiagen, Hilden, Germany) for SNP genotyping.

Bisulfite modification was performed on 1 μg DNA with MethylEasy Xceed (Human Genetic Signatures, North Ryde, Australia) according to the manufacturer's instructions, using two elutions of 50 μL, giving a final theoretical concentration of 10 ng/μL, or 500 ng was modified using the 96-well Epitect kit (Qiagen) eluting in a final volume of 40 μL. For DNA modified with MethylEasy Xceed, 1 μg Universal methylated DNA (Chemicon, Billerica, Mass.) was used as the methylated control and second round WGA product was used as an unmethylated control. For Epitect modified DNA, methylated and unmethylated DNA controls from Qiagen were used.

Genotyping by High Resolution Melting Analysis

The primers used for genotyping the rs16906252SNP were 5′-CTTTGCGTCCCGACGCCCGCAG-3′ and 5′-CCCAGACACTCACCAAGTCGC AAA-3′. PCR cycling and MS-HRM was performed on the Rotor-Gene Q (Qiagen) in 100 μl PCR tubes with a final volume of 20 μl, containing 200 nmol/l of each primer, 200 μmol/l of each dNTP, 0.5 U of HotStarTaq DNA polymerase (Qiagen) in the supplied PCR buffer containing 2.5 mmol/l MgCl₂, 5 μmol/l SYTO9 (Invitrogen, Carlsbad, Calif.), and 10 ng of bisulfate-treated DNA.

The initial denaturation (95° C., 15 minutes) was followed by 45 cycles for of 20 seconds at 95° C., 30 seconds at 68° C. (67° C. for SNP genotyping and addition of m13 tags prior to sequencing), 30 seconds at 72° C.; one cycle of 1 minute at 95° C., 72° C. for 1.5 minutes and a HRM step from 65° C. to 90° C. rising at 0.2° C. per second, and holding for 1 second after each stepwise increment. 5ng DNA was used as template for genotyping, and either 10 or 12.5 ng DNA was used for methylation studies. All reactions were performed in duplicate.

Sequencing was performed using Big Dye Terminator v3.1 chemistry (Applied Biosystems, Foster City, Calif.) according to the manufacturer's instructions. The initial denaturation (96° C., 1 minute) was followed by 30 cycles of 10 seconds at 95° C., 5 seconds at 50° C. and 4 minutes at 60° C. The sequencing products were purified by ethanol precipitation and separated on a 3100 Genetic Analyser (Applied Biosystems).

Primers for Methylation Analysis and Sequencing

Primer (and probe) sequences for the COL2A1 conversion control assay and the MGMT MethyLight assay have been previously published (Virmani A K, Tsou I A, Siegmund K D, et al. Cancer Epidemiol Biomarkers Prey 2002; 11(3):291-7). The primers used for the first MGMT SMART-MSP assay were 5′-TTcggatatgTtgggaTagTTcgc-3 and 5′-gAAcgtcgAAacgcaaaAcg-3′ where the capitalised bases correspond to non-CpG cytosines. The assay had multiple non-CpG cytosines but no CpG cytosines in the region between the primers. The primers used for MGMT SMART-MSP SNP genotyping were: 5′-cgattTagaTaTtTaTTaagtcgTaaacg-3′ and 5′-cgcccgcaAAtcct cgcgAtAcg-3′ where the capitalised bases correspond to non-CpG cytosines. The primers used for amplifying templates for sequencing were: 5′-ggTtgTTaTCGtTTCGagggagagTt-3′ and 5′-cgcgCCCcgaATATaCTaaaAC-3 followed by 5’-TGTAAAACGACGGCCAGTggTtgTTaTCGtTTCGagggagagTt-3′ and 5′-CAGGAAACAGCTATGACagcgCCCcgaATATaCTaaaAC-3′ to tag the amplicon with m13. The m13 primers used for sequencing were: 5′-TGTAAAACGACGGCCAGT-3′ and 5′ -CAGGAAACAGCTATGACC-3′

Results

A panel of 89 normal individuals (Red Cross blood donors) were genotyped for the rs16906252 SNP. We detected 77 CC homozygotes, 11 CT heterozygotes and one TT homozygote.

DNA prepared from the mononuclear cell fraction of peripheral blood was tested for MGMT methylation by two sensitive methodologies capable of detecting low-level methylation: SMART-MSP and MethyLight. SMART-MSP is an MSP based methodology that uses real time amplification for quantification and high resolution melting analysis to quality control the results. This allowed us to exclude false positive results due to significant incomplete conversion. MethyLight is an MSP based methodology that uses a TaqMan probe to quantify the results and to select against false positive amplification (Eads C A, Danenberg K D, Kawakami K, et al. Nucl Acids Res 2000; 28(8):e32-).

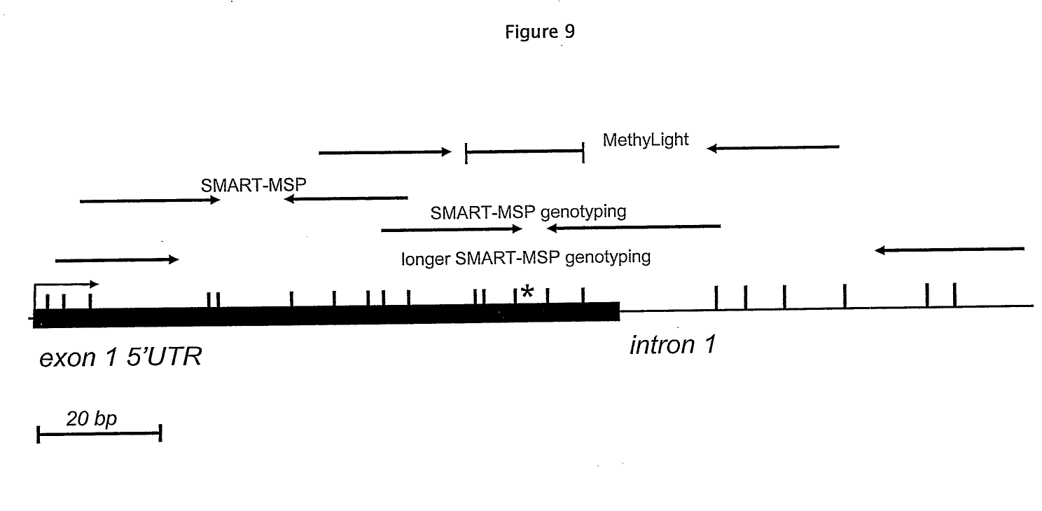

The SMART-MSP assay region was located in the 5′ UTR, immediately upstream of the rs16906252 SNP and is shown in FIG. 9. The TT homozygote, 6 of the 11 CT heterozygotes and 5 of the 77 CC homozygotes showed mosaic methylation using the assay (Table 2a) (p>0.0001). The levels of methylation ranged from 0.1% to 9.7% (Table 3). The level of methylation detected in the 3 CC individuals was low (0.1 or 0.2%). Very similar results were obtained using a MethyLight assay (Table 2b). The region assayed and its relationship to the SMART-MSP assay is shown in FIG. 9. None of the CC homozygotes had detectable methylation using the MethyLight assay (Table 2b).

To determine whether there was preferential methylation of either of the alleles in the heterozygous individuals, a second SMART-MSP assay was designed using primers that flanked the rs16906252 SNP (FIG. 9). The assay was targeted to the antisense strand containing the A/G alleles, as the CIT SNP is lost after bisulfate conversion converts the C to a T in the sense strand. This assay gave methylation results that were very similar to the first assay and the MethyLight assay though there was variation in the estimated level of methylation (Tables 2c, 3).

TABLE 2a
Summary of data from SMART-MSP assay 1

| | Methylated | Unmethylated | Total |
|---|---|---|---|
| CC | 3 | 74 | 77 |
| CT & TT | 7 | 5 | 12 |
| Total | 10 | 79 | 89 |

Fisher's test p = 1.17 × 10⁻⁵

TABLE 2b
Summary of data from MethyLight assay

| | Methylated | Unmethylated | Total |
|---|---|---|---|
| CC | 0 | 75 | 75 |
| CT & TT | 4⁺ | 5 | 9 |
| Total | 4⁺ | 80 | 84 |

Fisher's test p = 6.5 × 10⁻⁵
⁺Two not included came up as false positives
Five are missing because they were not done - ran out of DNA.

TABLE 2c
Summary of data from SMART-MSP assay 2 (SNP flanking)

| | Methylated | Unmethylated | Total |
|---|---|---|---|
| CC | 2 | 75 | 77 |
| CT & TT | 7 | 5 | 12 |
| Total | 9 | 80 | 89 |

Fisher's test p = 3.7 × 10⁻⁶

Table 3. Concordance of methylation data across experiments. The results of three separate assays are summarised. All the CT and TT samples are shown. In addition, all CC samples that displayed methylation are shown. The SMSP column shows results from the first SMART-MSP assay, the SMSP+SNP column shows results from the second SMART-MSP assay that contains the SNP. The column also shows the allele (antisense) detected by HRM and sequencing. “-” denotes no methylation detected, and ND denotes not done.

| Sample | Genotype | SMSP | SMSP + SNP | MethyLight |
|---|---|---|---|---|
| 1 | CC | 0.2% | — | — |
| 2 | CC | 0.1% | — | — |
| 3 | CC | 0.1% | — | — |
| 4 | CC | — | 0.2% G | — |
| 5 | CC | — | 0.1% G | — |
| 6 | TT | 0.1% | 3.1% A | 0.2% |
| 7 | CT | 9.7% | 4.5% A | 3.1% |
| 8 | CT | 8.4% | 2.5% A | 0.2% |
| 9 | CT | 0.8% | 0.1% A | 1.2% |
| 10 | CT | 0.4% | 1.7% A | 0.2% |
| 11 | CT | 0.2% | 0.1% A | 0.1% |
| 12 | CT | 0.1% | 0.1% A | ND |
| 13 | CT | — | 0.8% A | — |
| 14 | CT | — | — | — |
| 15 | CT | — | — | — |
| 16 | CT | — | — | ND |

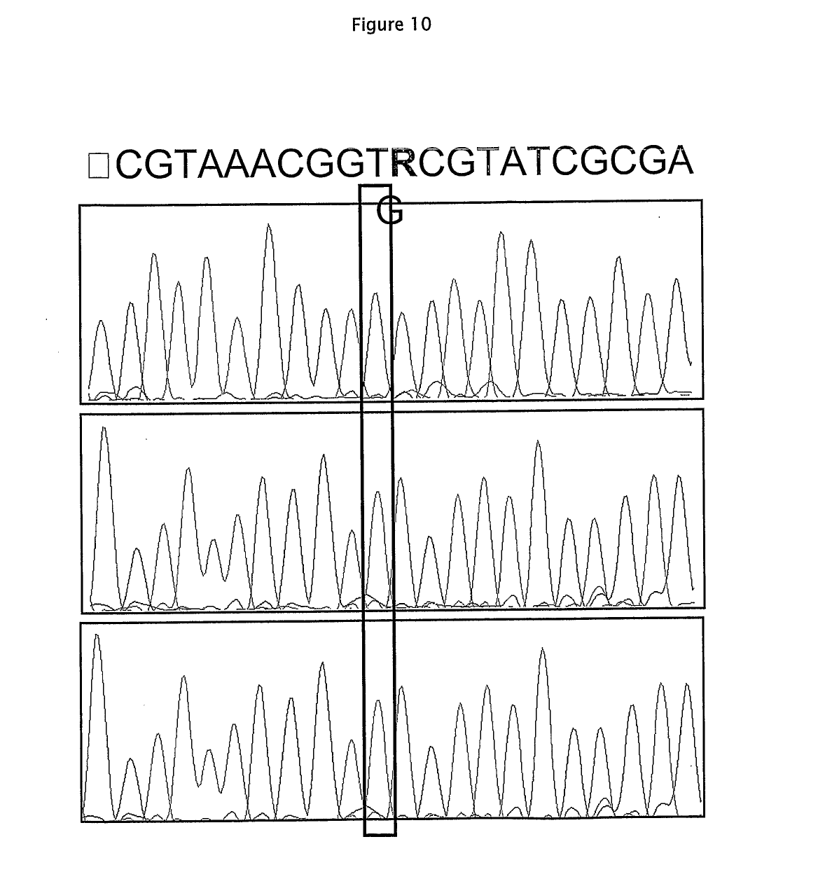

This second SMART-MSP assay containing the SNP allowed us to determine which allele was methylated as only methylated alleles were amplified. High resolution melting analysis showed that all six heterozygotes with detectable levels of promoter methylation displayed methylation of the T allele only. This result was confirmed by bisulfite sequencing of the antisense strand of a longer MSP amplicon that also included the SNP (amplicon shown in FIG. 9). All products of the heterozygotes showed only the A allele confirming that only the T allele was methylated in all of the heterozygotes (FIG. 10).

1. A method for detecting and determining nucleic acid methylation in a sample, said method comprising producing a methylation specific PCR (MSP) product from the sample and analysing the methylation in the sample in combination with analysis of melting of the MSP product inclusive of the region between primers used in the PCR.

2. A method according to claim 1 wherein the nucleic acid is DNA.

3. A method according to claim 1 which determines incomplete conversion of the nucleic acid sample when producing the methylation specific PCR (MSP) product.

4. A method according to claim 1 which determines heterogeneous methylation of the DNA sample when producing the methylation specific PCR (MSP) product.

5. A method according to claim 1 wherein the DNA sample is treated with a modifying agent to convert unmethylated cytosine bases to uracil.

6. A method according to claim 1 wherein the nucleic acid sample is amplified in the presence of primers having at least one cytosine at or near a 3′ end allowing methylation specific amplification.

7. A method of evaluating DNA methylation in a sample, the method comprising (i) reacting the DNA with an agent that differentially modifies methylated cytosine and non-methylated cytosine to produce modified DNA, (ii) amplifying the modified DNA by methylation specific PCR to produce amplified DNA wherein the methylation specific primers are selected such that the sequence between the primers includes a region of known sequence variation and/or at least one cytosine nucleotide, and (iii) subjecting the amplified DNA to melting analysis.

8. A method according to claim 1 wherein the analysis of melting is High Resolution Melting (HRM) analysis.

9. A method according to claim 1 wherein the methylation specific primers are selected such that there are only non CpG cytosines between the primers.

10. A method according to claim 1 wherein the methylation specific primers are selected such that there are both CpG and non CpG cytosines between the primers.

11. A method according to claim 1 wherein the methylation specific primers are selected such that there are only CpG cytosines between the primers.

12. A method according to claim 1 wherein the methylation specific primers are selected such that there is a sequence variant between the primers.

13. A method according to claim 1 wherein the methylation specific PCR (MSP) amplification is monitored in real time.

14. A method according to claim 1 wherein the DNA sample is amplified in the presence of a HRM compatible dye.

15. A method according to claim 14 wherein the HRM compatible dye is a DNA double stranded intercalating dye.

16. A method according to claim 14 wherein the dye is present at saturating levels and does not substantially interfere with the PCR.

17. A method according to claim 14 wherein the dye is a fluorescent dye.

18. A method according to claim 14 wherein the dye is selected from the group consisting of SYTO®9, EvaGreen™ and LC Green®.

19. A method according to claim 1 wherein the nucleic acid sample is derived from a biological sample which contains nucleic acid is selected from the group consisting blood, sputum, urine, plasma, serum, cells, fresh and archival tissues, saliva, tears, vaginal secretions, lymph fluid, cerebrospinal fluid, mucosa secretions, peritoneal fluid, amniotic fluid, ascitic fluid, fecal matter, body exudates and combinations thereof.

20. A method according to claim 19 wherein the tissue is selected from the group consisting of eyes, intestine, kidneys, brain, heart, prostate, lungs, breast, liver, histological slides and combinations thereof

21. A method for prediction of predisposition to, diagnosis of, prognosis of, monitoring of, or determining the likely response to clinical intervention of a genetic and/or chronic, and/or neoplastic disorder, the said method comprising determining a level of nucleic acid methylation of a nucleic acid sample from a patient wherein the level of methylation is determined by a method according to claim 1 and concluding the predisposition, diagnosis, prognosis, monitoring or likely response to treatment of the disorder from the level of methylation.

22. A method according to claim 21 wherein the disorder is cancer.

23. A method according to claim 21 wherein the cancer is breast cancer.

24. A method according to claim 21 wherein the disorder is any chronic disease selected from the group comprising cardiovascular disease, inflammatory conditions, and degenerative disease.

25. A method according to claim 21 wherein the disorder is an imprinting disorder selected from the group consisting of Prader-Willi syndrome, Angelman syndrome, and Beckwith-Wiedemann syndrome.
